(19) United States
(12) Patent Application Publication
(10) Pub. No.: US 2024/0293786 A1
(43) Pub. Date: Sep. 5, 2024

(54) SMALL VOLUME LIQUID MIXING AND DISPENSING SYSTEM AND METHOD

(71) Applicant: SCINOGY PRODUCTS PTY LTD, Mount Martha, Victoria (AU)

(72) Inventors: David JAMES, Fingal, Victoria (AU); Stephen WILSON, Kew, Victoria (AU); Ian FITZPATRICK, Burrill Lake, NSW (AU)

(21) Appl. No.: 18/575,263

(22) PCT Filed: Jun. 30, 2022
(86) PCT No.: PCT/AU2022/050686

§ 371 Date: Dec. 28, 2023

(30) Foreign Application Priority Data
Jun. 30, 2021 (AU) 2021902000

(51) Int. Cl. B01F 35/71; B01F 23/45; B01F 35/21

(52) U.S. Cl. CPC B01F 35/717611; B01F 23/45; B01F 35/21112; B01F 35/718051

(57) ABSTRACT

Embodiments provide a liquid handling system arranged for preparation of small volume liquid formulations and dispensing these into output vessels. The system can be utilised for a variety of low volume liquid formulation preparation applications, with cell therapies being one example application.

### TECHNICAL FIELD

The technical field of the invention is a mixing and dispensing of low volume liquid formulations with high accuracy, for example for application in preparing therapeutics or cell therapy liquid formulations.

### BACKGROUND

Regenerative medicine and advanced cell therapies are emerging medical therapeutic technologies that build on manipulation of live, human derived cells to create constructs, deliver immunogenic responses or stimulate repair responses in the patient body. While some of these techniques can deliver many doses to multiple patients from a single source of cell (allogeneic products,) there is growing recognition that processing and delivering cells derived from the patient or matched donor is safe and efficacious. To produce patient or matched donor specific cell products (autologous products) typically requires small batch processing. Batch processing can include mixing of fluids to manipulate concentration of cells and composition of carrier solutions. As these liquid formulations are used for medical therapy, high accuracy and an aseptic environment is required. Current processes typically require significant manual intervention and checking. (For example, manually calibrating scales and weighing bags of liquids to ensure accuracy.) Accuracy may also be limited by the tolerances of equipment (such as scales) used for performing checking.

Considering cell therapies, formulation, fill and finish is the final fluidic process conducted on cell based therapeutic products. Cells have a limited life outside the body, and often it is impractical to apply cell therapy to a patient immediately. It is therefore typically necessary to store cell based therapeutic products, for a least a short period of time. These products are commonly stored by cryogenic freezing. A “cryoprotectant” media is commonly mixed with the cell product as part of the formulation process. The cells can become unstable and have a short time to remain viable in cryoprotectant media until they are frozen. There is a limited time window from exposure to the media before freezing. If this window of stability for the cryoprotectant if missed this may result in increased cell death. This is particularly undesirable for autologous therapies, as typically cell counts are low, and every cell can be considered precious.

As a formal medicinal product, quality assurance scrutiny is intense throughout this process. Samples of the incoming material are used to direct the formulation actions such as dilution and cryo-preservative addition. Samples of the formulated material can be taken to verify the formulation process. Samples of the final product are taken to verify cell dose and complete characterisation assays for the active cell product. This information is carefully reviewed before the batch of output material can be released for administration to a patient. Autologous cell products relate to very small batches of product where input material is drawn from the patient or donor selected specifically for a patient. The number of doses from such a batch is commonly quite low. Because of the many manipulations, the time pressure and the small number of doses, these actions have been conducted manually. To meet good manufacturing practice (GMP) requirements for medical products, a second person will be monitoring and recording every step into a batch record document. The batch record is reviewed as part of the product release activities.

Manual processing of the liquids requires open processing, where the cell product is exposed to the working environment and relies on the skills of the operator to sustain an aseptic process. Manual handling also takes time. Given cells have a relatively short time period within which they must be used or safely frozen to remain viable, manual processing to produce the therapeutic may take up a significant proportion of this time. Thus, reducing the remaining time in this window to deliver the therapy for maximum efficacy.

Another problem with manual processing is the human trait to do things subtly differently on a progressive basis to improve the activity. It has been demonstrated that different people will consistently conduct the same actions quite differently. This can be impactful for cell products, for example:
Mixing—manual mixing of the cell product for homogeneity of the suspension can be highly variable.
Sample isolation—related to mixing, separating a small sample of product that reflects bulk of a homogeneously mixed suspension is quite difficult.

Although some automated systems are emerging, substantial manual intervention is still required to achieve the accuracy and consistency required for medical applications.

There is a need for systems and automated methods which produce low volume, high accuracy concentrations and compositions for liquid formulations.

### Definitions:

The following description refers to the terms Qualification and Verification. Formal quality assurance use of these words should be understood.

Qualification is a planned and documented activity that robustly confirms a process, equipment or cell product meets the stated requirements for the product. Qualification is a state that must be sustained by ongoing measurement or verification.

Verification is measurement of a process to determine it is performing acceptably well to meet the needs of the qualified process.

Calibrate is to adjust or check an instrument (such as a measuring device or precision pump) so that it can be used in an accurate and exact way.

### SUMMARY OF THE INVENTION

According to one aspect there is provided a liquid handling system comprising:
a reusable subsystem; and
a replaceable subsystem,
the reusable subsystem comprising:
   a peristaltic pump;
   a valve assembly comprising a plurality of valves;
   two or more bubble sensors each arranged to detect bubbles in a fluid path;
   a system controller configured to receive input from the bubble sensors, control operation of the peristaltic pump, and control operation of the valve assembly in accordance with a programmed processing protocol; and
   case housing the peristaltic pump, and valve operation assembly, and the single use, replaceable subsystem comprising:
   a fluid path manifold comprising one or more fixed geometry fluid paths, at least one of the fluid paths being configured for engagement with the valve assembly whereby fluid paths can be selectively opened or closed by operation of the valve assembly, the fixed geometry of at least one fluid path being arranged to sit proximate the bubble sensors when secured in the housing such that the bubble sensors can identify bubbles within the fluid path;
   a pump tube configured to enable operable engagement between the peristaltic pump and the fluid paths to cause fluid flow within the manifold by operation of the peristaltic pump;
   a plurality of a plurality of liquid input ports each configured for connection to respective liquid supply components for delivery of respective liquids to the one or more fluid paths;
   at least one gas inlet connected to at least one of the one or more fluid paths to enable gas to enter the fluid path; and
   at least one outlet port in fluid communication with the one or more fluid paths to dispense fluid,
   the replaceable subsystem providing a closed environment for mixing and dispensing liquid formulations,
   wherein the controller determines a volume of liquid in one of the fluid paths based on action of the peristaltic pump and inputs from at least one bubble sensor associated with the respective fluid path.

In an embodiment of the system the combination of fixed geometry fluid manifold and bubble sensor arrangements enable measurement of a known volumes in at least one region within the fluid paths and the controller utilises the known volume measurement to calibrate the peristaltic pump.

In an embodiment the peristaltic pump is automatically calibrated by the controller.

In some embodiments the peristatic pump is dynamically calibrated during execution of the one or more processing protocols.

In some embodiments, based on bubble sensor input, the controller utilises the measurement of known volume to verify the volume of dispensed products.

In some system embodiments bubbles in fluid lines are used to separate small volumes of liquid and volume of each small volume of liquid is verified in at least two different regions in the flow paths using bubble sensor data from each respective region.

In an embodiment of the system the verified volume data includes dispensed volume data.

In an embodiment the gas inlet can be an air inlet. In an embodiment the air inlet comprises a sterile filter.

In some embodiments the system controller is further configured to determine, based on particle count in a liquid sample being processed, a dose formulation and number of doses to output, and controlling the system to mix the determined formulation and dispense the determined number of doses.

In an embodiment the system controller includes an interpolation engine adapted to mathematically resolve dose and formulation variables based on target values and ranges for the formulation variables, particle count, and variable prioritisation rules.

Another aspect provides a method of calibrating a peristaltic pump within a liquid handling system as described above, the method comprising the steps of:
introducing a volume of liquid into a flow path having a known volume between a first bubble sensor and a second bubble sensor;
introducing gas into the flow path, such that the volume of liquid is preceded and succeeded by a gas bubble;
operating the peristaltic pump to cause the volume of liquid to proceed through the flow path proximate the first bubble sensor to enable identification of a transition from liquid to bubble and recording the peristaltic pump position at the transition;
operating the peristaltic pump to cause the volume of liquid to be drawn through the known volume flow path to the second bubble sensor to enable identification of the same transition from liquid to bubble by the second sensor and recording the peristaltic pump position at the transition; and
calculating volume of fluid displaced for each index of the peristaltic pump based on the recorded positions and known fluid path volume.

It should be appreciated that to perform this calibration method it is not necessary to know the actual volume of the liquid being drawn through the fluid path. As the calibration is based only on identification of one edge of the volume of fluid, knowledge of this volume is not required.

Some embodiments provide a method for determining a volume between two bubble sensors. For example, to determine the volume of a measurement loop between two bubble sensors. This method can also be used to calibrate the peristaltic pump. This method can be used to initially determine the volume of a measurement loop between two bubble sensors. This method can also be used to verify the volume of a measurement loop between two bubble sensors.

The steps for determining a volume of a tube between two bubble sensors comprises the steps of:
a) controlling introduction of an initial volume of liquid into the tube,
b) operating the peristaltic pump to progress the initial volume of liquid such that an edge of the fluid is identified proximate a first bubble sensor,
c) controlling introduction of further liquid into the tube until liquid is detected by the second bubble sensor, and
d) operate the peristaltic pump to transfer the total volume of liquid, comprising the initial volume of liquid and further volume of liquid into an external vessel.

Optionally a further step may be executed to e) remove the external vessel holding the total volume of liquid. The total volume can be measured to externally verify the volume of the tube between the two sensors. For example, the external vessel into which the liquid is transferred may be weighed before and after transfer and the liquid volume based on the difference between these weights and a known density (mass/ml²) of the liquid. By storing the volume of liquid representing the tube volume in an external vessel, this is available for quality control purposes or external verification. Knowledge of the volume of the tube between the two bubble sensors enables calibration of other aspects of the system, including the peristaltic pump and tubing within the manifold.

Controlling the volume of liquid introduced into the tube can include measuring the volume of liquid introduced as this is introduced into the tube.

Another aspect provides a method of performing a mixing operation in a liquid handling system as described above, the method comprising the steps of:
a) operating the peristaltic pump to cause fluid flow through fluid paths;
b) actuating one or more valves to control selection and direction of fluid flow through the fluid paths;
c) monitor fluid flow through the fluid paths using the bubble sensors and determine fluid volumes based on fluid detection by at least one bubble sensor and operation of the peristaltic pump;
d) responsive to determining a target volume of the fluid has passed a bubble sensor, actuating at least one valve to introduce a bubble into the fluid flow path behind the target volume of the fluid and direct flow of the fluid within the fluid paths, including directing flow of the target volume of fluid to a mixing reservoir; and
e) repeat steps a) to d) for one or more further fluids and further target volumes whereby the fluids are mixed within the mixing reservoir.

The mixing method can further comprise the step of:
f) actuating one or more valves to recirculate the mixed fluid from the mixing reservoir through the fluid paths and back to the mixing reservoir.

The mixing method can further comprise the step of:
g) actuating one or more valves to cause the mixed fluid to flow though one or more fluid paths to an outlet and dispense a target volume of the mixed fluid based on operation of the pump and mixed fluid flow detected by a bubble sensor.

A target volume dispensed can be a sample volume of the mixed fluid. For example, a sample to test the mixture composition, before dispensing production volumes of the mixed fluids.

According to another aspect there is provided a reusable subsystem of a liquid handling system,
the reusable subsystem comprising:
   a peristaltic pump;
   a valve assembly comprising a plurality of valves;
   two or more bubble sensors each arranged to detect bubbles in a fluid path;
   a system controller configured to receive input from the bubble sensors, control operation of the peristaltic pump, and control operation of the valve assembly in accordance with a programmed processing protocol; and
   case housing the peristaltic pump, and valve operation assembly,
   wherein the pump, valve assembly and bubble sensors are arranged to engage with a fluid path manifold comprising one or more fixed geometry fluid paths, whereby fluid paths can be selectively opened or closed by operation of the valve assembly, at least one fluid sits proximate each of the bubble sensors when secured in the housing such that the bubble sensors can identify bubbles within the fluid path, and the pump engage with a pump tube configured to enable operable engagement between the peristaltic pump and the fluid paths to cause fluid flow within the manifold by operation of the peristaltic pump,
   wherein the controller determines a volume of liquid in one of the fluid paths based on action of the peristaltic pump and inputs from at least one bubble sensor associated with the respective fluid path.

According to another aspect there is provided a replaceable subsystem of a liquid handling system, the replaceable subsystem being configured to engage with a reusable subsystem comprising a peristaltic pump, a valve assembly comprising a plurality of valves, two or more bubble sensors each arranged to detect bubbles in a fluid path, a system controller configured to receive input from the bubble sensors, control operation of the peristaltic pump, and control operation of the valve assembly in accordance with a programmed processing protocol; and a case housing the peristaltic pump, and valve operation assembly, the replaceable subsystem comprising:
a fluid path manifold comprising one or more fixed geometry fluid paths, at least one of the fluid paths being configured for engagement with the valve assembly whereby fluid paths can be selectively opened or closed by operation of the valve assembly, the fixed geometry of at least one fluid path being arranged to sit proximate the bubble sensors when secured in the housing such that the bubble sensors can identify bubbles within the fluid path;
a pump tube configured to enable operable engagement between the peristaltic pump and the fluid paths to cause fluid flow within the manifold by operation of the peristaltic pump;
a plurality of a plurality of liquid input ports each configured for connection to respective liquid supply components for delivery of respective liquids to the one or more fluid paths;
at least one gas inlet connected to at least one of the one or more fluid paths to enable gas to enter the fluid path; and
at least one outlet port in fluid communication with the one or more fluid paths to dispense fluid,
the replaceable subsystem providing a closed environment for mixing and dispensing liquid formulations,
wherein the controller determines a volume of liquid in one of the fluid paths based on action of the peristaltic pump and inputs from at least one bubble sensor associated with the respective fluid path.

### BRIEF DESCRIPTION OF THE DRAWINGS

An embodiment, incorporating all aspects of the invention, will now be described by way of example only with reference to the accompanying drawings in which

FIG. 1 is a representative block diagram of a system embodiment;

FIG. 2 illustrates a basic example of the fluid transfer strategy that forms the basis for the system operation;

FIG. 3a is a representative block diagram of a control system 300 for an embodiment of the system;

FIG. 3b is a representative block diagram of a control system 301 for an embodiment of the system;

FIG. 4a is a basic representation of the equipment used for the pump calibration process;

FIG. 4b illustrates steps of a pump calibration process;

FIG. 5 is a flowchart of an example of a pump calibration process;

FIG. 6 shows an example of an embodiment of the system;

FIG. 7 is a schematic of fluid paths of a single use kit and associated valves and bubble sensors, also representing the peristaltic pump of the example of FIG. 6.;

FIG. 8 illustrates steps of a process for calibrating a “known volume” loop with an external reference;

FIG. 9 shows an example of additional equipment that may be used for calibration of the known volume loop to an external reference.

FIG. 10 shows a table of tube calibration measurement results.

### DETAILED DESCRIPTION

Embodiments provide a liquid handling system arranged for preparation of small volume liquid formulations and dispensing these into output vessels. The system can be utilised for a variety of low volume liquid formulation preparation applications, with cell therapies being one example application.

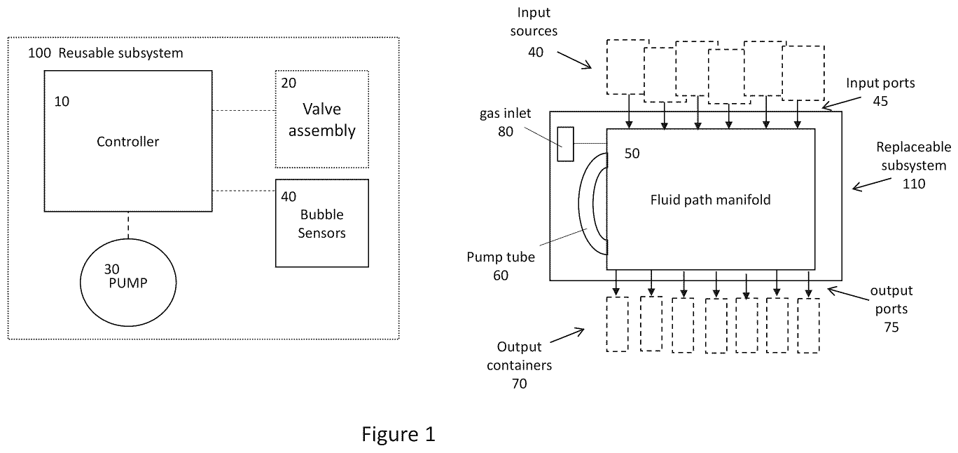

FIG. 1 shows a representative block diagram of an embodiment of the liquid handling system, the system comprises a reusable subsystem 100 and a replaceable subsystem 110. The reusable subsystem 100 comprises a controller 10, a valve assembly 20, a peristaltic pump 30 and at least two bubble sensors 40. The valve assembly 20 comprises a plurality of valves each configured to engage with a fluid path of fluid path manifold 50 of the replaceable subsystem 110. The bubble sensors 40 are arranged along at least one fluid path to detect presence or absence of liquid in the fluid path. These components are arranged within a housing that is arranged to operably receive the replaceable subsystem.

The replaceable subsystem 110 provides a closed environment for mixing and dispensing liquid formulations. The replaceable subsystem 110 includes a fluid path manifold 50 comprising one or more fixed geometry fluid paths, a flexible pump tube 60 operably engages with the peristaltic pump whereby operation of the peristaltic pump causes flow of fluid within the fluid paths. A plurality of liquid input ports 45 are provided, each configured for connection to respective liquid input sources for delivery of respective liquids to the one or more fluid paths. For example, the liquid input sources may be liquid supply components such as bags of input liquids with tubes to connect to the input ports, or the liquid input source may be an external system. At least one outlet port 75 in fluid communication with the one or more fluid paths is also provided to dispense fluid to one or more output containers, or to another system. In an embodiment a set of output containers may be incorporated into the replaceable subsystem.

The replaceable subsystem also includes a gas inlet 80 connected to at least one fluid path to enable gas to enter the fluid path. In an embodiment the gas inlet 80 includes a sterile filter and allows entry of filtered air to the fluid path. In other embodiments the gas inlet may be configured for connection to a gas cannister. The gas inlet allows gas to enter the fluid path, causing a bubble in the fluid path.

In embodiments of the system the replaceable subsystem is a single use kit comprising the peristaltic pump tube and a manifold of tubes accessed through valves supported in a carrier frame that controls the kit geometry when it is placed into the instrument (the reusable subsystem). The valves and bubble sensors are arranged in the housing of the reusable subsystem such that they align with the tubes in the manifold that form the fluid paths. The volume of fluid contained within the tubing between the bubble sensors can therefore be consistent. One configuration of the fluid path has demonstrated a total variation between replaceable subsystems of 0.05 ml over 3.2 ml. (1.6%)

The combination of operation of the peristaltic pump and actuation of the valves can control drawing of liquid from the input sources and movement of fluids through the fluid paths. Fluid transfers are managed by a peristaltic pump and the use of air to chase blocks of fluid through the lines. This strategy relies on the fluid lines being sufficiently small in diameter that surface tension of the fluid holds the blocks of fluid together. This enables mixing and dispensing to be performed as functionally closed processes. The controller is programmed to control the operation to perform specific mixing and dispensing processes as described below.

The controller can be programmable, for example implemented using a microprocessor and memory which can be programmed with software, to execute mixing and dispensing processes as described below. Other embodiments may use programmable hardware components, such as programmable logic controllers or field programmable gate arrays, which can be programmed to perform specific mixing and dispensing processes, such embodiments may include supporting software to enable programming of the programmable hardware. Alternatively, embodiments may be implemented using dedicated circuitry or an application specific integrated circuit (ASIC) designed to control execution of specific processes. It should be appreciated that an embodiment where the reusable subsystem includes a microprocessor and memory, enabling customising of mixing and dispensing processes using software is advantageous for research purposes and also for individualised therapies where customisation for each individual patient may be required.

Embodiments of this system can be utilised to perform functions one or more of:
mix controlled volumes of fluids with each other;
isolate samples of the mixed cell suspension;
prepare “doses” of cells and carrier fluid
and dispense controlled volumes to output vessels

Embodiments of the system can include:
means to self-verify calibration of the pump calibration used to control all fluid transfers.
Monitoring of fluid transfers in a way that provides verification of volumes transferred independent of the controlling function.
Means to determine and provide externally verifiable data regarding a volume of tubing between two sensors.

These features enable reconciliation of the fluids contributing to the final medicinal product formulation and doses.

As discussed in the background section automated systems are being developed to automate at least some steps for preparation of liquid formulations, and which complete the operations within bags and tubes creating a functionally closed process environment. This eliminates the risks of open processing. Automated systems can also create the opportunity to address other process risks associated with manual processing, such as inconsistencies and errors due to operator variation.

Automated processing now places the onus for volume precision into the equipment rather than the operator. Although the example given above relates to cell therapies, these problems can also apply for preparation of other types of therapeutic and non-therapeutic liquid formulations, particularly for small volumes.

Another advantage of processing automation is that of increased speed, including speed to product freezing, this reducing the time cells are required to remain viable before being frozen. Similarly, the amount of time the cells are mixed with cryoprotect before freezing is reduced, thereby reducing cell death.

A further advantage of automation is accuracy. Embodiments of the presently disclosed system enable accurate manipulation of very small volumes of liquid formulations. In particular the system incorporates several self-calibrating and/or self-verifying operations which support precision manipulation of fluids, in particular for automatically preparing treatment doses. These will be discussed in further detail below.

### Control of Fluid Transfers

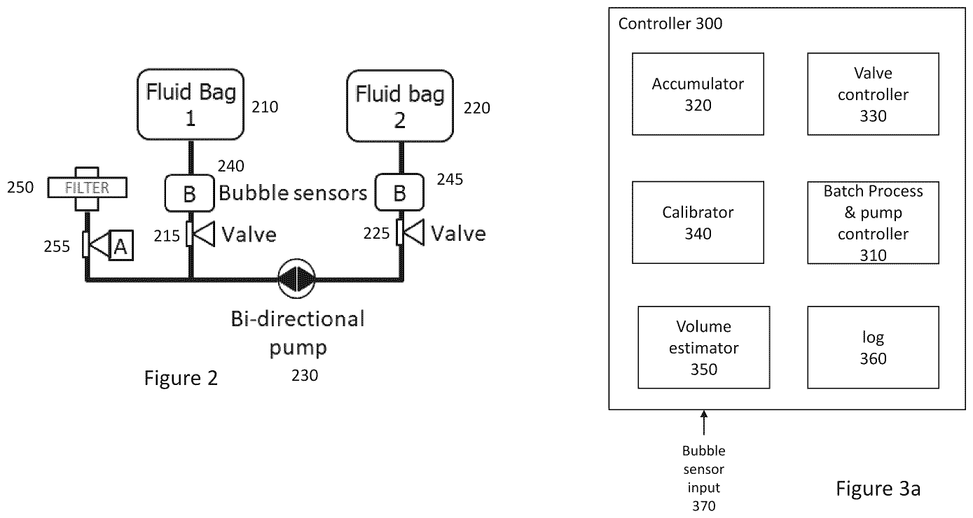

FIG. 2 illustrates a basic example of the fluid transfer strategy that forms the basis for the system operation.

The peristaltic pump 230 draws fluid past a control valve 240 from bag 1 210 and pushes it to bag 2 220, with control valve 245 open for the fluid to enter bag 2 220. Once the target volume has been drawn from bag 1 210, the control valve 240 for bag 1 is closed and a valve 255 is opened to enable air to be drawn in through a sterile filter 250. The pump continues to push the fluid into bag 2 220 using the air.

Fluid volume accuracy is managed by detailed control of the pump rotation and a known peristaltic pump volume delivery per revolution. The pump tube and pump geometry combine to create a pump calibration with the units of millilitre per revolution of the pump. Calibration of the pump can be performed automatically, and dynamically as is described in further detail below.

Each fluid control valve 240, 245 also has a bubble sensor 215, 225 associated with it. Bubble sensors 215, 225 detect the presence or absence of fluid inside the tube by means of differential ultrasonic impedance across the tube. Other types of bubble sensors may also be used, for example optical sensors.

A primary process risk for transferring fluid by peristaltic pump is whether there is in fact fluid being transferred. The bubble sensors monitor the fluid in the tube as the fluid transfer occurs. The bubble sensors send signals to the controller indicating the state of the fluid in the tube proximate the bubble sensors, thus the controller is informed of passage of liquid and air bubbles through the tubes. The volume of fluid that has moved through the tube past a bubble sensor can be calculated based on the fluid flow rate, which is related to the pump operation. The control system uses this information to determine the volume that has passed through each valve. So as the fluid transfer occurs, the fluid being accumulated in a bag can be calculated from the pump movement when the bubble sensor indicates there is fluid.

The peristaltic pump is a positive displacement type pump that has a rotor with “rollers” or “wipers” which press against a flexible fluid tube to cause the flexible tube to be pinched closed, rotary motion of the pump rotor causes the roller to move along the tube, thereby moving pinch point of the tube forcing fluid ahead through the tube. Each rotation will move the same volume of fluid through the tube, and the rotation of the rotor can be indexed such that a fluid volume moved by a partial rotation can be known and controlled based on the rotational positioning of the rotor. In particular the fluid volume moved for each pump rotation or each rotor index progression will be the same. Thus, volume moved by the pump can be known and controlled, based on the pump indexing, and thereby fluid flow rate be controlled by pump rotational speed. By knowing the fluid flow rate and monitoring the time for a body of fluid to pass by a bubble sensor the volume of the body of fluid can be calculated. Even more simply, instead of a fluid flow rate, the volume of a body of fluid can be calculated from a pump index (or rotation) count required to move the volume of fluid past a bubble sensor, as each count increment represents a known constant volume of fluid. For this method a controller increments a counter for each index increment of pump rotation. The count between start and end of the fluid volume, as detected by the bubble sensor, is directly proportional to the volume moved. The count can be simply multiplied by the volume movement for each rotational index of the pump to determine the fluid volume. Thus, accuracy of fluid volume calculation can be independent of time. Using this method also enables accurate fluid volume calculation when pump rotation speed may be variable, as only the rotational index count is required. Using a rotation index count for calculating moved volume may be particularly advantageous for very small volume mixing processes where required fluid volumes (for example to input for mixing) may be less than one rotation of the pump.

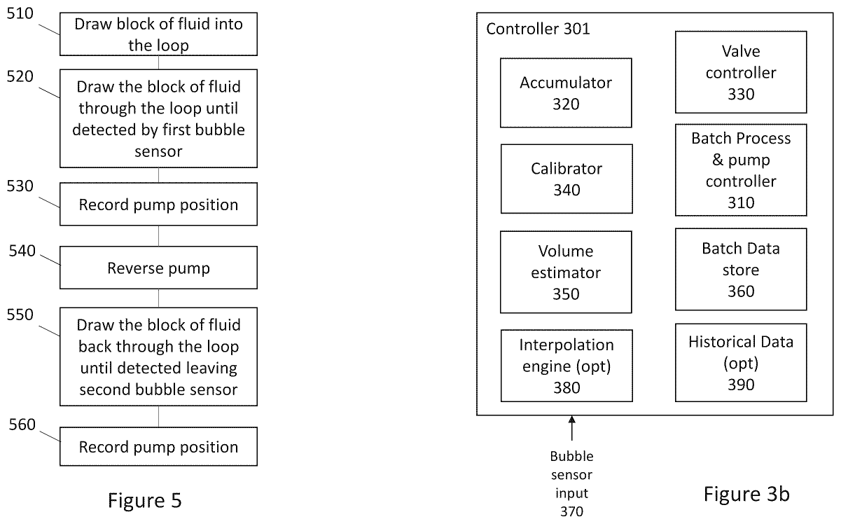

FIG. 3a is a representative block diagram of a control system 300 for an embodiment of the system, this block diagram is representative of the processing modules which may be implemented in software and/or hardware components. The control system 300 includes a batch process and pump controller 310 which is the main process controller for the particular batch protocol, an accumulator module 320 which calculates and tracks the volume of liquids and other formulation parameters during the batch processing process, a valve controller 330 which responsive to instructions from the batch process controller 310 actuates valves to control fluid flow during the batch processing process, a calibrator 340 to automatically calibrate the peristaltic pump, a volume estimator 350 to estimate volume of fluid based on the bubble sensor inputs 370, and a log 360 for logging information in relation to the batch processing.

The accumulator 320 tracks the liquid volumes drawn from and delivered to each reservoir or input source used in the batch processing, for example monitoring the volume of liquid drawn from one fluid bag and delivered to a mixing reservoir (which may be a fluid bag or other vessel optionally engaged with components to promote mixing) as well as tracking the volume in the mixing reservoir. The accumulator, can also estimate formulation concentration states at different stages of the processing based on the accumulated volume data.

Returning to the example of FIG. 2, the fluid is drawn from bag 1 210 so the accumulator will reduce the estimated volume in bag 1 210 as the pump draws down the target fluid while the bubble sensor 240 is wet. Then for bag 2 220 the accumulator similarly recognises fluid being delivered when the bubble sensor 245 for bag 2 220 is wet. The fluid can be finally driven through to bag 2 220 with air. The bubble sensor 245 recognises the air so the volume is not accumulating even though the pump is still active. This method has proven robust with 6 sigma tolerance 0.4 ml for volume from 2 ml to 50 ml.

It should be appreciated that using this method, volume for the same body of liquid is calculated based on bubble sensor data acquired in two different parts of the system, in the example of FIG. 2, during draw down from the bag 1 210 and during delivery to bag 2 220, thus, comparisons of the calculated volumes from both parts of the system enables internal verification/validation this robust method for accurately calculating the volumes delivered. The consequence, and the first claim of this invention is independent verification of the fluid volume transferred from both the source and destination vessels where bubble sensors are provided.

Where a final patient dose volume is delivered to a vessel, while the pump is controlling the volume dispensed, the accumulator measurement at the source bag, and the accumulator measurement at the destination vessel are independently determining the volume of liquid and not air that was drawn and delivered. This being based on the flow rate or rotational index count, which is a function of the pump rotation, and the bubble sensor output—sensing the start and end of the volume of fluid as this moves past the sensor due to operation of the pump. Close agreement between these measurements and the target delivery volume systematically verifies the fluid transfer step.

This method can be employed to measure the input product volume for the process. A common step is to draw down from a bag of input cell suspension and transfer it to a second bag configured for mixing the formulated product. The bubble sensor directed volume accumulators record the fluid drawn out of the input bag and received by the mixing bag. If the two accumulators do not agree within a defined tolerance, (commonly 0.1 ml but may vary between 0.05-0.7 ml depending on the application) the control systems returns the input material and repeats the transfer until the recorded volumes are within the tolerance.

### Pump Calibration Verification

The above technique for calculation and verification of liquid volume relies on confidence in the pump calibration, and indeed it would be normal practise to include a calibration protocol for a peristaltic pump medicinal product dispensing process. A known problem with peristaltic pumps is that the flexible tube which engages with the pump has an initial “warming up period” where the tube properties, and hence pumping behaviour, will change until the tube's flexibility stabilises and the pumping behaviour becomes constant. For example, the tube may be less flexible initially and therefore the rate of deformation and pumped volume changes as the tube becomes more flexible. For large batch processing or long periods of operation the difference in pumped volume caused by this initial phase may be within allowed tolerance for the specific application, or a specific initialisation protocol may be used to “warm up” the system before calibrating the pump. However, neither are suitable for low volume processing.

However, in small batch autologous processing, accurate calibration of the pumping is essential. Embodiments of the system include pump calibration functionality.

The self-verification capability of embodiments of the system is completed by means to verify the pump calibration as part of a set-up procedure. This calibration can also be performed periodically to verify or adjust the calibration of the pump.

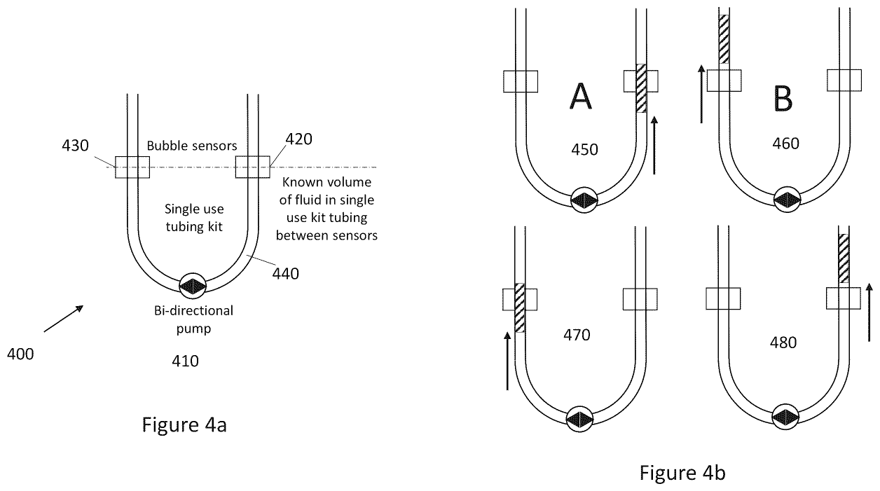

FIG. 4a is a basic representation of the equipment used for the pump calibration process, representing the portion of the instrument 400 that includes the peristaltic pump mechanism 410 and bubble sensors 420, 430 associated with control valves (not shown) for opening closing the fluid tubes. The instrument operates with a single use kit comprising the peristaltic pump tube 440 and a manifold of tubes (not shown) accessed through the valves which are supported in a carrier frame that controls the kit geometry when it is placed into the instrument. The volume of fluid contained within the tubing between the bubble sensors can therefore be consistent for each single use set. A variation of 0.05 ml has been demonstrated over 3.2 ml nominal volume. The volume of the tubing, and therefore fluid contained within the tubing, between the bubble sensors can therefore be known and used in calculations during processing. As discussed above a combination of finely controllable pumping bubble sensors enables accurate calculation of fluid volumes within the system for automation of mixing protocols. Key to the accuracy of fluid volume calculations is precise calibration of the peristaltic pump. Peristaltic pumps have manufacturers calibration data, however for use with very small volumes pumping accuracy is critical and therefore it is desirable to be able to verify the pump calibration. A calibration protocol for a peristaltic pump can be included in a setup procedure, for example a self-verification procedure for verifying pump calibration.

A process for calibrating the pump is illustrated in FIG. 4b and the flowchart of FIG. 5. The volume of a fluid tube between two bubble sensors is known. This can include the pump engagement loop 440. A first step 510 of the measurement process draws in a small block of fluid into the fluid path 440. The state 450 in FIG. 4b illustrates the fluid block is pumped forward through the tube until the first bubble sensor 420 detects it 520, shown in FIG. 4b as state 450. The pump 410 position is recorded 530 at the point where the first bubble sensor 420 detects the block of fluid in the tube 440.

The pump 410 is then reversed 540 to draw the fluid block back through the tube 440 until the fluid block is detected 550 leaving the second bubble sensor 430, shown in FIG. 4b as state 460. The pump position is recorded at this second position 560. Based on the known tube volume between the two bubble sensors, the volume for each pump rotation (or index) can be calculated from the number of rotations (or partial rotations) to move one edge of the fluid from one bubble sensor to the next. This can be checked against the manufacturer calibration data. This process can be repeated (or performed) in the opposite direction illustrated as states 470 and 480 in FIG. 4b and the values for both the forward and backward directions compared with each other and the manufacturer calibration data. If the pump index volume calculated from the test measurements is within a pre-set tolerance (for example 1% of manufacturer's calibration values), then the pump calibration setting may be considered verified. This data may also be recorded in a process log. Calculated values outside the tolerance or discrepancy between forward and reverse operation measurements may cause the calibration verification to fail and be repeated. Data regarding number of verification attempts and results may also be logged.

Pump position is recorded with an accuracy within 0.002 ml. This test method eliminates the volume of the fluid block from the calculation of the volume of fluid moved with each index rotation of the peristaltic pump, as only the one edge of the fluid block needs be detected by the bubble sensors 420, 430.

This process can be repeated (or performed) in the opposite direction illustrated as states 470 and 480 in FIG. 4b. In an embodiment the calibration method cycles the block of fluid back and forward recording the measurements monitoring for consistent readings. The calculation relies on the bubble sensor function. If either the forward or reverse pump direction measurements do not agree with instrument setting by more than a pre-set tolerance (for example 1%), an automated sequence can be executed to capture additional samples of the pump calibration measurement. Statistical data derived from the progressive samples is monitored to determine when adequately consistent results have been obtained (for example, 6×standard deviation<0.2 ml for example). The calibration factor is then automatically updated and reported to the process run record. For example, a reason for changing measurements and need to perform and iterative calibration process may be a “warm up” phase for the flexible tubing of the pump.

Once consistent reading are established, the pump calibration in the forward direction and the reverse direction are estimated and can be compared to the formally calibrated value initially stored in system memory by the manufacturer or otherwise maintained by the instrument. Data relating to the calibration verification process (for example, iterations and measurements) as well as calibration verification results can also be reported to the process run record (for logging in memory with other data for the batch process). Assuming the results are within the predefined tolerance, the pump calibration has been verified.

Embodiments may also perform dynamic recalibration or re-checking of the calibration during processing. It should be appreciated that as volumes of the tubes between each bubble sensor is known or can be calculated based on the fixed geometry of the manifold, any fluid block moving between two bubble sensor may be used to detect the fluid block and compare a calculated pump rotation index for the edge of the fluid block to pass the next bubble sensor with the actual pump rotation index to verify the pump operation. If a variation is detected, the amount of variation may be due to one or more changes in the system. For example, change in ambient operating temperature may affect fluid pressure, changing flexibility of the pump tube may alter the efficiency of the pump, or an error or leakage in the system may have occurred. A diagnostics function may identify whether the variation can be attributed to a drift which is within operating tolerance or can be rectified by adjusting calibration. If the diagnostics indicates a potential error the process may be stopped, and/or an operator alerted to the error.

### Known Volume Verification

The method of automatically calibrating or verifying calibration of a peristaltic pump within a functionally closed system takes advantage of a consistent volume created by the tubing of a single use kit controlled in a carrier frame which in turn is controlled by geometric features on the process equipment. Qualification of the peristaltic pump calibration requires measurement of the fluid volume between the two bubble sensors to ensure that this volume remains known. Further, as the tubing between the bubble sensors is part of the single use subsystem, some minor variation of the volume (within manufacturing tolerances) is to be expected between kits.

Qualification of the pump calibration action requires a means to measure the volume of fluid in the controlled measurement loop independent of the tubing, instrument, sensor function or installation variability.

The volume determination process uses the same features to isolate the volume of fluid the measurement system uses for calibration. Verification of the volume can be performed across multiple different single use kits. Further, different instruments provide the statistical confidence for the volume being measured, (using the resources deployed for the calibration itself,) and hence the calibration determined from it.

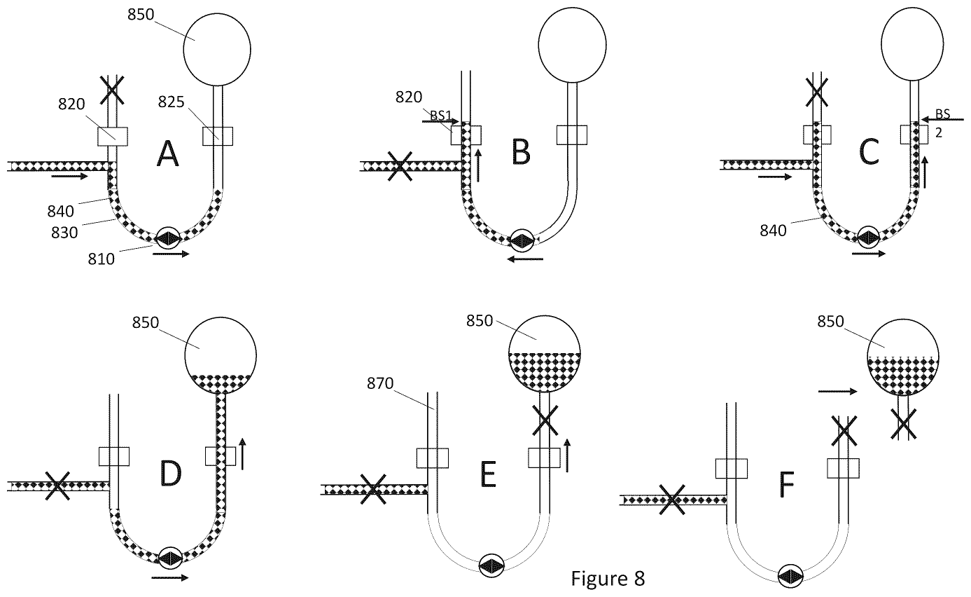

The method uses automated control of valves and pumps interacting with the fluid detection sensors to isolate the controlled volume to a vessel that can be removed from the kit for precise weight measurement. An example of the process is illustrated in FIG. 8. In a first step A. a controlled volume of fluid 840 is transferred into the measurement loop 830. The peristaltic pump 810 can be operated to draw the fluid forward through the tube 830, until at step B. the Fluid is backed up to and triggers the first bubble sensor 820 to be detected by the first 820 bubble detection sensor. In this step the peristaltic pump 810 is operated in reverse in step B.

In step C, additional fluid 840 is transferred into measurement loop until second fluid sensor 825 triggered. It should be appreciated that the volume of fluid between these sensors represents the “known fluid volume” as discussed above. In step D the pump is operated forwards and the fluid representing measurement loop volume transferred into external vessel 850. The medium used to push the volume of liquid through the fluid tube can be filtered air, as described above. In a step E. the external vessel is isolated form the resto of the fluidic assembly. For example, a valve may be used to isolate the external vessel 850. Optionally the external vessel can be removed for calibration of loop fluid measurement. To enable the external vessel to be removed a separation point can be provided at a point that is predictable from a mass perspective. Measuring the weight of the external vessel before and after the addition of the fluid enables a mass-based calculation of the tube volume. For example, where the liquid has a known weight, Xg/ml then from the difference in weight of the vessel between empty and filled, the volume can be readily calculated.

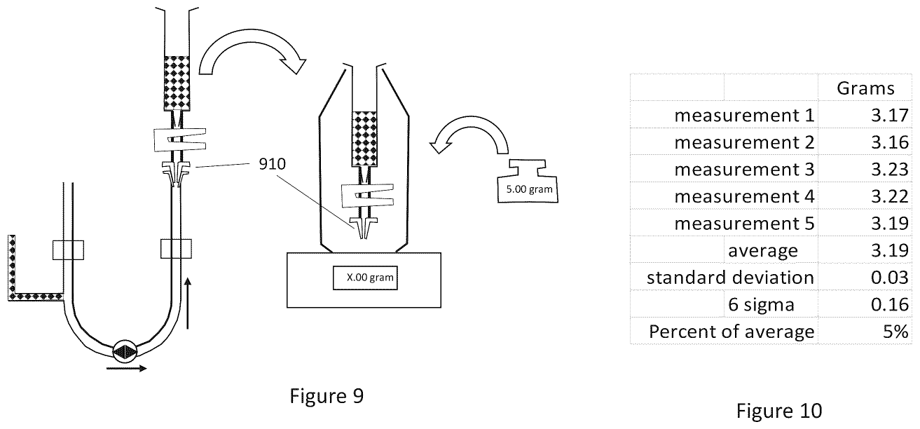

FIG. 9 shows an example of additional equipment that may be used for calibration of the known volume loop to an external reference. In this example the external vessels are connected to the liquid handling system using luer connections 910 between the external vessel and the system being measured. Other options are envisaged. An advantage of luer connectors is that these are a standard and commonly used medical device connector with high reliability. Which can also be removed and reconnected. In an alternative embodiment, the external vessel may be part of the single use kit, connected via a tube, and disconnected once full by cutting the tube.

FIG. 10 shows an example of a table of measurements of the calibration loop using the above described method. These results display six sigma measurement variation of 5% for the loop volume.

An advantage of embodiments of the described system is that processing accuracy can be validated within the system, without requiring manual intervention. This is enabled by virtue of the closed system, fixed geometry of the fluid paths in the fluid path manifold, and use of bubble sensors in a plurality of positions along the fluid paths. Use of the bubble sensors and air gaps between means that liquid volumes can be estimated at more than one area in the system, based on bubble sensors output. Thus, the same block of liquid may have its volume estimated multiple times in different areas of the fluid paths, and if comparison of these multiple estimations shows concurrence this indicates accurate measurement. Thus, the volume of a liquid block dispensed to an output container or bag has already been verified within the system, reducing reliance on manual methods such as weighing.

Formulation, fill and finish is the final fluidic process conducted on cell based therapeutic products. The system described herein has the advantages of enabling strategy and methods for process self-verification by the automated equipment completing the formulation, fill and finish operations.

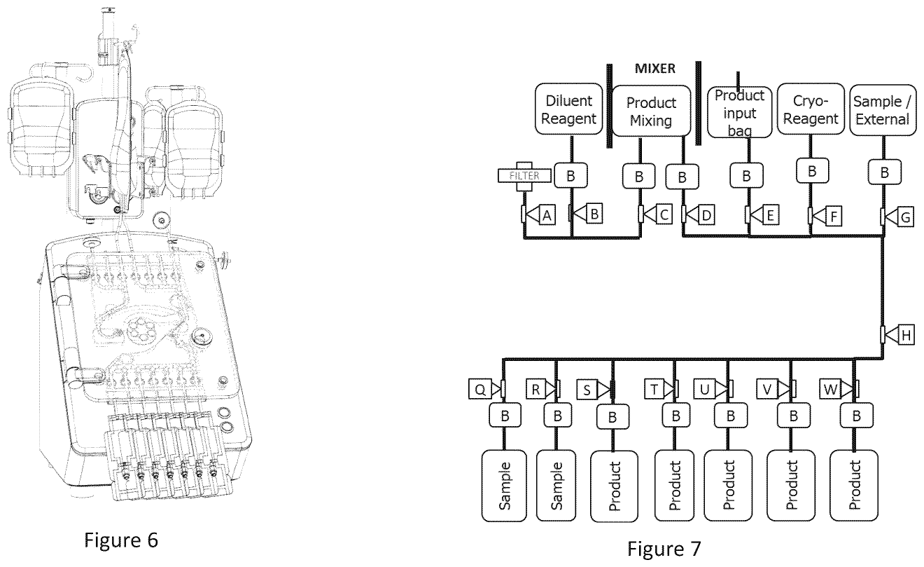

An example of an embodiment of the system is shown in FIG. 6, with a schematic diagram of the key functional components shown in FIG. 7. In this embodiment of the instrument the reusable subsystem 100 includes a mixer and hangers for supporting fluid bags above the case housing the control system, peristaltic pump, valve assembly and bubble sensors. The mixer can mechanically massage the bag to promote mixing of liquids held within in the bag. In some embodiments tubes may also be provided either directly from the system or via one or more of the input bags to another system, such as a centrifuge. In the embodiment shown in FIG. 6, a single use subsystem is shown mounted within the case. Each of the bags can be connected to fluid input paths of the single use kit manifold, and a plurality of small bags are connected to outputs of the fluid paths, into which treatment volumes of the formulation can be dispensed.

An embodiment may also include a recirculation function, which circulates fluid through the fluid paths of the manifold, optionally combining with further input fluids during this circulation, and back to the mixing bag. This circulation may occur more than once either for combining with additional input fluids or as a mixing step. This provides an ability to create an homogeneous suspension of the fluid by combining the bulk mixing of the bag and the detailed circulating of that suspension through the single use kit tubing. A sample can then be drawn from that recirculating stream with a highly representative example of the bulk of the homogenous suspension, for example for testing purposes or dispensing in smaller volumes.

FIG. 7 is a schematic of the fluid paths of the single use kit and associated valves and bubble sensors, also representing the peristaltic pump.

The embodiment shown in FIGS. 6 and 7 has an input bag on the right and diluent reagent on the left to facilitate rinsing of input bag. For reagent dispensing, the liquid from the reagent bag is dispensed past bubble sensor when valve F is open, and action of the pump can cause this liquid to be moved through the fluids paths to the mixing bag when valve D is open. Valves can be selectively actuated to allow inputs from each of the input sources to enter the fluid flow paths, and operation of the pump controls how much liquid is drawn into the system from each input source. Operation of the pump controls the volume of liquid drawn from each of the input sources, the volume being determined, as discussed above, based on the known fluid path geometry and operation of the pump, as the volume pumped for each revolution of the pump has been calibrated.

Operation of the pump also delivers the input liquids to the mixing bag. The liquid can be pushed through the fluid paths using air due to narrow dimensions of the fluid flow paths and surface tension of the liquid causing the input liquid to pass as blocks through the fluid paths. An air inlet with a sterile filter is provided such that when valve A is open air is drawn into the fluid paths by operation of the pump. This air is used to push blocks of liquid around the fluid paths. The system may be programmed to control many different variations of movements of selectively controlled volumes of fluids between reservoirs in order to mix precise formulations.

Air gaps or bubbles can also be used to divide dispensable quantities of liquid in the fluid paths. For example, when dispensing a plurality of samples of a mixed formula, the air valve A can be actuated to allow a bubble to form in the fluid line between each dispensing volume sample. The volume of each sample can be calculated as it is drawn down from the mixing bag and subsequently verified as the block of liquid passes bubble sensors. Thus, the system provides self-verification capability.

To consider the advantages of the disclosed system it is worth considering the prior art alternatives. Primary process control for formulation fill and finish is fluid transfer volume. Commercial prior art systems in the market at the time of filing rely on mass measurement to determine the volume of fluid that has been transferred through weighing of hanging bags, or with the trays of platform scales.

Response from the weighing system is dependent on an offset or tare adjustment and a scale factor. The tare is managed by determining when it should occur in the process and working around that. The scale factor is less volatile but given the absence of secondary information there is a risk it could be in error every time the system is used. Verification of all the weighing devices should therefore be part of every batch activity.

By having weighing systems at the source and destination vessels, each transfer can be verified. A substantial scaling error by one of the weighing devices or other interference will be evident in the failure of the two measurements to match. Another problem with weighing batch inputs and outputs, which is a systematic problem with these systems is interference. Since closed systems are linked with tubing that shifts as bags fill and empty, this limits the weighing precision that can be achieved, and can add to weighing related reliability issues. (Where unexplained changes occur between two weighing devices.)

In contrast the system embodiments described herein build on the inventors' experience with use of peristaltic pump tubing and the control of the pump rotation, and advantageously provides an internally verifying system.

The inventor's system relies on a known pump tube behaviour, affected by the pump tube material and geometry, and the geometry of the pump rollers and squeeze applied to pump tube. There are many potential variables, but these systems are widely used for precision fluid delivery in large scale fill and finish systems. The pump tubing is manufactured to high standards for this purpose.

Nevertheless, use of the peristaltic pump as the primary fluid volume control exposes the process to the risks:
1. The calibration of the pump tube is erroneous or has been corrupted.
2. Accidental pumping of air or bubbles in the fluid line rather than just liquid.

In embodiments of the present system ultrasonic bubble sensors are used. These devices detect the presence or absence of fluid inside a tube by virtue of the ultrasonic sound impedance. A threshold is set to detect bubbles in the tube. These devices are commonly used with intravenous infusion pumps to protect patients from receiving fine bubbles of gas instead of fluid. In this application, the bubble sensor threshold can be quite coarse since we are only interested in gross gaps in the fluid stream. The air gaps are deliberately used to divide specific portions of liquid in the fluid paths.

The ability to utilise bubbles for separating specific volumes of liquids and ability to verify these specific volumes within the system enables high accuracy mixing and tracking of the respective concentrations of components in mixed formulations. This data is logged during the mixing process and can be utilised for quality control and qualification of output products. The ability to accurately verify volumes and concentrations internally can reduce the burden of manual verification. This can have the advantageous effect of improving processing and/or treatment delivery times. Particularly for delicate samples, reduction in manual handling steps and shortened preparation time, may have significant quality and treatment outcome advantages.

Some embodiments do not include consideration of additional sources of interference such as temperature control. Temperature measurement is however a good example of in-process measurement that needs to be carefully considered to ensure it can be qualified robustly. Invariably the only practical way to qualify temperature measurement is to calibrate the measurement system from time to time and demonstrate that the frequency of calibration events is sufficient to maintain an acceptable accuracy. In a manual calibration system, this is a cumbersome, manual exercise which can introduce errors.

An advantage of the system described herein is internal verification functionality. This can manifest in:

In-process pump tube calibration verification-Pump tube calibration compares measured volume to controlled volume in kit assembly.

Input product volume measurement—Product volume transferred from the input bag is measured and can be used for batch data

Sample volumes independently recorded and removed from product pool

Product volume pool—input, diluent and cryo-buffer is managed as batch data

Volumes dispensed to output bags measured independent of dispense controller and recorded for review. Poor correlation captured in record and can be highlighted for inspection.

Embodiments of the system can also provide reports such as Batch closeout includes volume reconciliation report, based on data logged during execution of the mixing and dispensing process.

### Automated Formulation and Dose Dispensing

The above discussed advantages of self-calibration and verification of the system make the system suitable for automation of at least significant parts of a cell therapy dose preparation process. Potentially a full dose preparation process may be automated using embodiments of the disclosed system.

For context, the following paragraphs provide an overview of an example process for preparing an autologous cell treatment. As an initial step, cells are harvested or collected from a patient (or donor matched to the patient for an allogenic treatment). These cells are kept alive and may be cultivated to multiply (also referred to as expansion) in a culture medium, for example for a few hours to around 10 days. This may also involve specific treatment of target cells before or during the culture period. The cells possibly with at least some of the culture medium can be transferred into a centrifuge system where the target or “good” cells can be separated from dead cells, non-target cells and other waste products. For example, a reverse flow centrifuge may be used to separate the target cells from other cells or waste particles, the culture medium may also be flushed away through dilution with another carrier medium. For example, due to variation in size and weight between target cells and other components (dead cells, non-target cells, cell fragments or other waste particles) target cells will cluster together where forces (fluid flow and centrifugal forces) acting on these cells are balanced within the centrifuge chamber. The reverse flow centrifuge is operated to separate the target cells and concentrate these in a fluidised bed. It should be appreciated that until these target (or good) cells are the basis for the cell therapy treatment, however the number of cells obtained is unknown. The number of cells will vary from patient to patient due to many variables. The variables which influence the number of cells can include patient biological factors, and variables acting on the culture process, for example, ambient temperature or pressure, concentrations in culture media components or surrounding atmosphere. The number of cells obtained can also be unpredictable. Cell counts may be very low, for example ranges can be from only one hundred thousand cells, one million cells, tens of millions of cells, to a few hundred million cells.

Once the target cells are separated from waste material these typically need to be formulated into doses for delivery to the patient. As culturing the cells takes some time and special equipment is required for isolating the target cells, typically this preparation takes place in a lab or other facility remote from the patient. The cells need to be preserved for transportation and delivery back to the patient. This is typically done by freezing in a mixture comprising a cryoprotectant, in individually administrable doses. Desired dose parameters can be defined for the therapy. These parameters may include dose volume and dose formulation (for example proportions and composition of carrier media, cryoprotectant), minimum number of doses required for treatment. However, the precise formulation composition may be variable based on cell concentration. Precise quantities required for the formulation is typically dependent on the number of cells in the dose. For example, a carrier solution may comprise is a mix of fluids which may include cryoprotectant. The cells for a dose are suspended in the carrier solution. The volume of carrier solution can be manipulated to dilute the concentration of cells for a dose. Permissible ranges, or thresholds, can be defined for treatments, for example a minimum cell count for a dose, associated cryoprotect volume range based on cell count, and ranges for any other formulation components. These components in the formulation may vary depending on the purpose of treatment. For example, a formulation of doses for stem cell therapy for cancer treatment may vary depending on the type of the cancer being treated.

The feasible number of doses will be dependent on the number of cells obtained for use in these doses. Thus, the precise dose formulation, and number of doses cannot be pre-set, but rather must be determined after the cells have been separated and a cell count can be performed. Cell count is conventionally performed by either manually or automatically count the number of cells in a small volume of fluid (for example 0.1 ml) providing a count of cells per ml. For example, a small sample of the input material (often referred to as the QC sample) is extracted and a cell count performed on this sample. (This may be manual or using an external cell counting system.) As the system can record or measure the volume of the input material, the total number of cells can be calculated by simple multiplication the total number of cells per millilitre with a determined volume of the suspension.

Once the cell count is performed, the potential number of doses feasible to produce from the total cells can be calculated. As the number of doses must be a whole number, it is likely that given a target or preferred number of cells for each dose there will be some residual cells. For example, a cell count determines that 60 million cells are obtained for use in treatment doses. Parameters stored in the system define a target cell count per dose of 40 million cells, with an allowable range of 25 to 50 million cells per dose. From the available 60 million cells it is only possible to formulate one dose of 40 million cells, leaving a residual of 20 million cells which is below the minimum dose threshold of 25 million cells. In such instances the options are:
1. Prepare one dose at 40 million cells and discard the remaining 20 million cells.
2. Reduce the cell count per dose to enable use of all cells, for example two doses of 30 million cells, or one dose of 35 million cells and a second dose of 25 million cells.
3. Increase the cell count for the once dose to 50 million to maximise the cells used and minimise the number of cells discarded to 10 million cells.
4. Increase the cell count for the one dose to 60 million to use all cells. This may be undesirable due to the dose being outside the allowable range.

It should be appreciated that options 2 and 3 appear to be the most desirable in the current circumstances, option 2 maximises cell utilisation and maintains doses within the define ranges. Option 3 minimises discarded cells within the defined dose ranges, and is therefore preferable over option 1. Further, as this option provides only one dose, it may be tolerable for this dose to be at the maximum allowable cell count, or even above as in option 4 for this treatment. As should be apparent determining the number of doses and quantity of cells per dose involves assessment of various trade-offs and constraints to determine the dose. Conventionally a clinician will decide on such dose compromises.

Once the dose for a treatment is determined, then further calculations are required to determine the formulation for each dose. Again, such calculations may include trade-offs/compromises and constraints. Currently decisions regard dose formulation and calculation of required volumes of the components to formulate the dose are made manually.

Clinicians may utilise a computer as a calculation tool, for example, using a spreadsheet to assist in performing these calculations. However, the process is still substantially manual and heavily dependent on the experience and knowledge of the clinician. The large number of variables involved make these calculations time consuming. It should also be appreciated that manual processing can introduce errors or inconsistencies. The process for manually calculating dose formulations can also take time—thereby extending the time cells are required to survive before doses are frozen.

An embodiment of the disclosed system is configured to enable automated calculation of dose formulations based on a cell count. An example of a control system 301 configured to perform dose calculations is illustrated in FIG. 3b, this system is similar to FIG. 3a having a batch process and pump controller 310 which is the main process controller for the particular batch protocol, an accumulator module 320 which calculates and tracks the volume of liquids and other formulation parameters during the batch processing process, a valve controller 330 which responsive to instructions from the batch process controller 310 actuates valves to control fluid flow during the batch processing process, a calibrator 340 to automatically calibrate the peristaltic pump, a volume estimator 350 to estimate volume of fluid based on the bubble sensor inputs 370, and a log 360 for logging information in relation to the batch processing.

The accumulator 320 tracks the liquid volumes drawn from and delivered to each reservoir or input source used in the batch processing, for example monitoring the volume of liquid drawn from one fluid bag and delivered to a mixing reservoir (which may be a fluid bag or other vessel optionally engaged with components to promote mixing) as well as tracking the volume in the mixing reservoir. The accumulator, can also estimate formulation concentration states at different stages of the processing based on the accumulated volume data.

In this embodiment the controller 301 further includes an interpolation engine 380 which can operate in real time to calculate dose parameters based on an input cell count. In this embodiment the controller stores mathematical formulae and algorithms for dose calculation, variables for dose calculation and variable attributes and values such as ranges or boundary conditions for each of the variables, and variable prioritisation data. The variables and associated attributes are typically based on treatment requirements and can include information such as:

Dose volume—with range or boundary conditions defining minimum and maximum volumes

Dose cell count—which may include one or more ranges specifying upper and lower limits, or thresholds.

Carrier formulation component variables and Cryoprotect details, including constraints, concentration ranges, and any cell concentration dependencies.

Each variable can have defined a target value and a permissible range. An interpolation engine utilises these defined target values and ranges to mathematically resolve the variables and determine the dose formulation for the given the cell count. The constraints may include threshold values for viable treatment, for example minimum cell count per dose, and minimum dose number. If insufficient cells are available to deliver a viable treatment the formulation may be stopped and an alert output to the clinician, so the clinician may then determine next actions.

The system also stores prioritisation rules which can be varied between batches. For example, a prioritisation may be 1. Maximise number of doses for the cell count, 2. Maximise cell count within a given range, 3. Round down to whole number of doses. 4. Whether or not a maximum or minimum range is able to be exceeded to avoid excess cells being discarded. The ranking of such prioritisation rules is variable and may be predefined by the clinician, based on the cell therapy. This prioritisation is utilised by the interpolation engine to mathematically resolve the variables and determine the dose formulation.

It should be appreciated that the target and ranges for each variable and prioritisation rules can be selected for each batch prior to the physical processing being started. These parameters, ranges and prioritisations may vary between batches. In some embodiments historical data 390 may be stored which includes previous batch data, including previously used parameters, ranges and prioritisations. In this embodiment a clinician or technician may select previously used settings based on batch type. In some embodiments the settings may be stored as profiles for batch types. For example, via user interface stored batch profiles may be looked up in a pick list or searched from data records.

Having the interpolation engine 380 integrated with the controller 301 of the fluid handling system has an advantage in that calculation of the dose formulation can be rapid, and once the formulation is resolved the controller can automatically prepare the doses in accordance with the determined formulation, using methods as described above.

In an embodiment the cell count is manually entered by a clinician or technician. In this embodiment a sample of the concentrated cells is extracted and counted, a cell count per millilitre is input to the system. Based on this value the controller is configured to determine the total cell count, based on the total volume of concentrated cells and the cell count. And utilise this value and batch dose parameters, ranges and prioritisations to mathematically determine an optimal dose formulation. The optimal dose formulation being resolved by the interpolation engine using an iterative process. This iterative process can be broadly characterised by the following steps:
A. Based on total cell count and target number of cells per dose (or target cell concentration and dose volume) determine integer value for number of doses and any residual.
B. If no residual, does integer value for total number of doses meet minimum dose number requirements. If minimum dose number is met continue to determine dose formula (step E). If minimum dose number is exceeded, and priorities preference additional doses, then continue to dose formula determination (step E). If minimum or target dose number is exceeded, and priorities preference increased dose concentration, then recalculate dose concentration (step C).
C. If there is a residual or minimum dose number is exceeded and increased dose concentration is preferred. Based on total cell count recalculate dose number based for an increased cell count per dose. If dose number and cell concentration are within allowable ranges, then continue to dose formula determination (step E).
D. If the minimum target dose number is not met in step A, and there is no residual, the system may, based on threshold requirements for the treatment the system may determine that no viable treatment can be formulated and output a warning message to the clinician or technician. Processing can be halted at this step to enable the clinician to input further instructions—for example, the cells may be output to be returned to a growth media or further examination. If minimum viable treatment thresholds are met, then the system may continue to determine the most preferable dose option based on the prioritisation data and number of cells available. Once a viable treatment dose number and cell count per dose is determined, the dose formulation can be determined (step E)
E. Formulate dose based on cell count per dose: the amount of cryoprotectant used in the dose formulation is based on the number of cells per dose, the dose formulation may also include other media or carrier fluids. The interpolation engine iteratively resolves variable values based on mathematical formulae and prioritisation rules to tailor the dose formulation for the cell count and treatment. This may involve varying the dose volume within a defined range as well as adjustment of relative concentrations of components of the formulation.

It should be appreciated that the process described above, in practice, may involve iterative calculations at each step, and/or steps may be iteratively repeated until an optimal formulation is determined. Once the formulation is determined (i.e. the ratios of components to be mixed for each dose, and dose volume), the system automatically performs the steps of mixing the formulation and dispensing the individual doses for freezing, using the process described above. This automation can significantly shorten the time between the cells being removed from the culture or growth media and being frozen in individual doses.

It should be appreciated that the automated calculation and subsequent dose preparation is enabled by the automatic calibration and verification of volumes within the system, which means the measurement of components for mixing into the formulation is accurate and reliable, similarly the output dose volumes are also accurate.

In some embodiments of the system a sample (a QC sample) is automatically output for manual cell count. It is also envisaged that embodiments of the system may be utilised for automated mixing and dispensing of doses where the cell count of an input concentration of cells or other particles is already known (for example, determined from another process outside the liquid formulation system) before the concentrated cells or particles in suspension are input to the system. The process for measuring volume of the concentrate, dose formulation calculation, mixing and dispensing can then be carried out as described above.

Embodiments of the system may also be configured for integration with a system capable of automatic determination of cell (or particle) count. Cell count data may be input to the formulation processing via a machine to machine communication interface between the systems, this may be a wired or wireless interface. The cell count can be based on detected characteristics of the concentration of cells in suspension. For example, optical properties (density, turbidity, spectrum) of the concentration of cells in suspension may be measured using an optical sensor. Other sensors may also be used, such as electrical sensors. Cell concentration may be estimated based on these characteristics and the type of cells.

The need to understand the particle count in a suspension is a common quality control measurement that is conducted for particle based therapeutic products since it is needed to direct the next steps of the process. Such particle counts are commonly achieved by taking a small sample of the dilute suspension in a total known volume, and an instrument or manual optical methods used to determine the particles in the small sample. The particle count in the total suspension volume is then extrapolated based on the small sample count. The problem is that this sampling and particle counting action requires the primary process to wait until that information has been acquired before proceeding. Further these methods for particle counting are subject to many influences that contribute to variation in the measurement determination, resulting in count variations that range +/−20%. Indirect observation of the entire particle population by a density sensor combined with an accumulation of verification data of the same product and process environment may provide adequate confidence to complete a process without in-process sampling.

An example of a reverse flow centrifuge system which may include functionality for automated cell count estimation is disclosed in the applicant's earlier patent applications publication number WO2019/140491 and WO2018/204992, such as system may be connected with the disclosed system via a centrifuge line (see FIG. 7) to enable integration of the functionality of the two systems. In this example, in counter flow centrifuge processing, the fluidized bed of particles created in the rotating chamber will be characterized by attributes of the particles-nominal external dimension or diameter, bulk density and external surface morphology that affect the stokes settling behaviour in the fluid media supporting the particles. Fluid media attributes include density and viscosity (both sensitive to temperature), and second order characteristics such as thixotropy or shear sensitivity of viscosity. Despite the complexity of these interactions, replication of a process with largely consistent input materials and operating conditions will deliver a consistent fluidized bed behaviour that results in consistent particle density in the fluidised bed when measured as a number of particles per unit volume (particles per ml for example). It should be appreciated that the density of the particles in suspension will be substantially similar between separate batches processed using consistent input materials and operating conditions but the volume of the fluidised may vary significantly between batches reflecting changes in the number of particles.

Embodiments of the centrifuge system in such instances may include a particle count estimation. In these embodiments the counterflow centrifuge controller is further configured to determine a particle count based on recovered concentrate volume, and particle density estimation for the concentrate based on particle characteristics and operating parameters. The particle density estimation can be based on empirical data, for example historical data from processing previous batches having input materials and operating conditions correlating with the current processing batch. For example, such data may be accumulated externally and input to the controller with the processing procedure data and parameters for the particular process being performed. Alternatively, the controller may be configured to monitor process execution and capture data characterising the particle density (for example density sensor outputs, particle count estimation or verified particle count data) for each executed process. In such embodiments the controller may store such data in a database or other data repository for look-up and comparison with a current processing event to identify one or more correlating previous/historical processing events and look up suspension characteristics to use for particle density estimation. Once the particle density is determined, the total cell count is calculated from the density and measured volume. This estimated cell density or cell count can be output to the controller 301 for use in the dose formulation process. In some embodiments a quality control sample (QC sample) may also be output for verification purposes. However, for processing (for example types of batches) where the system has a record of accurate automated cell count estimation, extraction of a QC sample may be omitted to maximise the number of cells available for formulation into doses.

It should be appreciated that the automated processing enabled by the system can significantly reduce the time between cells being removed from culture media (or the body) and frozen in therapeutic doses for delivery. Further, the ability to integrate with external systems, such as centrifuge systems, can increase the processing steps able to be autonomous, potentially further improving processing speed and consistency. Reduction in processing time can have significant impacts on cell viability and treatment outcomes. Integration of autonomous closed processing systems can also reduce risk of exposure to contaminates or environmental conditions. Thus, improvements afforded need not only be related to speed.

It will be understood to persons skilled in the art of the invention that many modifications may be made without departing from the spirit and scope of the invention.

It is to be understood that, if any prior art publication is referred to herein, such reference does not constitute an admission that the publication forms a part of the common general knowledge in the art, in Australia or any other country.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A liquid handling system comprising:
a reusable subsystem; and
a replaceable subsystem,
the reusable subsystem comprising:
a peristaltic pump;
a valve assembly comprising a plurality of valves;
two or more bubble sensors each arranged to detect bubbles in a fluid path;
a system controller configured to receive input from the bubble sensors, control operation of the peristaltic pump, and control operation of the valve assembly in accordance with a programmed processing protocol; and
case housing the peristaltic pump, and valve operation assembly, and the single use, replaceable subsystem comprising:
a fluid path manifold comprising one or more fixed geometry fluid paths, at least one of the fluid paths being configured for engagement with the valve assembly whereby fluid paths can be selectively opened or closed by operation of the valve assembly, the fixed geometry of at least one fluid path being arranged to sit proximate the bubble sensors when secured in the housing such that the bubble sensors can identify bubbles within the fluid path;
a pump tube configured to enable operable engagement between the peristaltic pump and the fluid paths to cause fluid flow within the manifold by operation of the peristaltic pump;
a plurality of a plurality of liquid input ports each configured for connection to respective liquid supply components for delivery of respective liquids to the one or more fluid paths;
at least one gas inlet connected to at least one of the one or more fluid paths to enable gas to enter the fluid path; and
at least one outlet port in fluid communication with the one or more fluid paths to dispense fluid,
the replaceable subsystem providing a closed environment for mixing and dispensing liquid formulations,
wherein the controller determines a volume of liquid in one of the fluid paths based on action of the peristaltic pump and inputs from at least one bubble sensor associated with the respective fluid path.

2. A system as claimed in claim 1, wherein the combination of fixed geometry fluid manifold and bubble sensor arrangements enable measurement of a known volume in at least one region within the fluid paths and the controller utilises the known volume measurement to calibrate the peristaltic pump.

3. A system as claimed in claim 2, wherein the peristaltic pump is automatically calibrated by the controller.

4. A system as claimed in claim 3, wherein the peristatic pump is dynamically calibrated during execution of the one or more processing protocols.

5. A system as claimed in claim 2, wherein based on bubble sensor input, the controller utilises the measurement of known volume to verify the volume of dispensed products.

6. A system as claimed in claims 1, wherein bubbles in fluid lines are used to separate small volumes of liquid and volume of each small volume of liquid is verified in at least two different regions in the flow paths using bubble sensor data from each respective region.

7. A system as claimed in claim 6, wherein the verified volume data includes dispensed volume data.

8. A system as claimed in claim 1, wherein the gas inlet is an air inlet.

9. A system as claimed in claim 8, wherein the air inlet comprises a sterile filter.

10. A system as claimed in claim 1, wherein the system controller is further configured to determine, based on particle count in a liquid sample being processed, a dose formulation and number of doses to output, and controlling the system to mix the determined formulation and dispense the determined number of doses.

11. A system as claimed in claim 10, wherein the system controller includes an interpolation engine adapted to mathematically resolve dose and formulation variables based on target values and ranges for the formulation variables, particle count, and variable prioritisation rules.

12. A system as claimed in claim 3, wherein the controller automatically calibrating the peristaltic pump by controlling the liquid handling system to:
introduce a volume of liquid into a flow path having a known volume between a first bubble sensor and a second bubble sensor;
gas into the flow path, such that the volume of liquid is preceded and succeeded by a gas bubble;
operate the peristaltic pump to cause the volume of liquid to proceed through the flow path proximate the first bubble sensor to enable identification of a transition from liquid to bubble and recording the peristaltic pump position at the transition;
operate the peristaltic pump to cause the volume of liquid to be drawn through the know volume flow path to the second bubble sensor to enable identification of the same transition from liquid to bubble by the second sensor and recording the peristaltic pump position at the transition; and
the controller calculating volume of fluid displaced for each index of the peristaltic pump based on the recorded positions and known fluid path volume.

13. A system as claimed in claim 12, wherein the controller determining determines a volume of a tube between two bubble sensors by:
a) controlling introduction of an initial volume of liquid into the tube,
b) operating the peristaltic pump to progress the initial volume of liquid such that an edge of the fluid is identified proximate a first bubble sensor,
c) controlling introduction of further liquid into the tube until liquid is detected by the second bubble sensor, and
d) operating the peristaltic pump to transfer the total volume of liquid, comprising the initial volume of liquid and further volume of liquid into an external vessel.

14. A system as claimed in claim 1, wherein the configured to performing a mixing operation by:
a) operating the peristaltic pump to cause fluid flow through fluid paths;
b) actuating one or more valves to control selection and direction of fluid flow through the fluid paths;
c) monitor fluid flow through the fluid paths using the bubble sensors and determine fluid volumes based on fluid detection by at least one bubble sensor and operation of the peristaltic pump;
d) responsive to determining a target volume of the fluid has passed a bubble sensor, actuating at least one valve to introduce a bubble into the fluid flow path behind the target volume of the fluid and direct flow of the fluid within the fluid paths, including directing flow of the target volume of fluid to a mixing reservoir; and
e) repeat steps a) to d) for one or more further fluids and further target volumes whereby the fluids are mixed within the mixing reservoir.

15. The system as claimed in claim 14, wherein the controller further performs the step of:
f) actuating one or more valves to recirculate the mixed fluid from the mixing reservoir through the fluid paths and back to the mixing reservoir.

16. The system as claimed in claim 14 further comprising the step of:
g) actuating one or more valves to cause the mixed fluid to flow though one or more fluid paths to an outlet and dispense a target volume of the mixed fluid based on operation of the pump and mixed fluid flow detected by a bubble sensor.

17. The system as claimed in claim 16, wherein the target volume dispensed is a sample volume of the mixed fluid.

18. A reusable subsystem of a liquid handling system,
the reusable subsystem comprising:
a peristaltic pump;
a valve assembly comprising a plurality of valves;
two or more bubble sensors each arranged to detect bubbles in a fluid path;
a system controller configured to receive input from the bubble sensors, control operation of the peristaltic pump, and control operation of the valve assembly in accordance with a programmed processing protocol; and
case housing the peristaltic pump, and valve operation assembly,
wherein the pump, valve assembly and bubble sensors are arranged to engage with a fluid path manifold comprising one or more fixed geometry fluid paths, whereby fluid paths can be selectively opened or closed by operation of the valve assembly, at least one fluid sits proximate each of the bubble sensors when secured in the housing such that the bubble sensors can identify bubbles within the fluid path, and the pump engage with a pump tube configured to enable operable engagement between the peristaltic pump and the fluid paths to cause fluid flow within the manifold by operation of the peristaltic pump,
wherein the controller determines a volume of liquid in one of the fluid paths based on action of the peristaltic pump and inputs from at least one bubble sensor associated with the respective fluid path.

19. A replaceable subsystem of a liquid handling system, the replaceable subsystem being configured to engage with a reusable subsystem comprising a peristaltic pump, a valve assembly comprising a plurality of valves, two or more bubble sensors each arranged to detect bubbles in a fluid path, a system controller configured to receive input from the bubble sensors, control operation of the peristaltic pump, and control operation of the valve assembly in accordance with a programmed processing protocol; and a case housing the peristaltic pump, and valve operation assembly, the replaceable subsystem comprising:
a fluid path manifold comprising one or more fixed geometry fluid paths, at least one of the fluid paths being configured for engagement with the valve assembly whereby fluid paths can be selectively opened or closed by operation of the valve assembly, the fixed geometry of at least one fluid path being arranged to sit proximate the bubble sensors when secured in the housing such that the bubble sensors can identify bubbles within the fluid path;
a pump tube configured to enable operable engagement between the peristaltic pump and the fluid paths to cause fluid flow within the manifold by operation of the peristaltic pump;
a plurality of a plurality of liquid input ports each configured for connection to respective liquid supply components for delivery of respective liquids to the one or more fluid paths;
at least one gas inlet connected to at least one of the one or more fluid paths to enable gas to enter the fluid path; and
at least one outlet port in fluid communication with the one or more fluid paths to dispense fluid,
the replaceable subsystem providing a closed environment for mixing and dispensing liquid formulations,
wherein the controller determines a volume of liquid in one of the fluid paths based on action of the peristaltic pump and inputs from at least one bubble sensor associated with the respective fluid path.
